SEQUENCE LISTING (19 sequences)

SEQ ID NO: 1 - DNA, 1896 bp, Trichoderma reseii
  misc_feature (1)..(1896): Trichoderma reseii glucoamylase gene
atgctactccaagcattcctttttctgttagcaggatttgctgccaaaatctctgctagacctggatcttcaggcttgtccgacgtcacaaaaagatccgtggatgattttatctctacagaaacacctattgcacttaacaatctcctgtgtaatgttggaccagatggttgtagagcattcggcacaagtgcaggcgctgttattgcttctccatctacaattgatccagactattactacatgtggacaagagactccgcccttgtgttcaaaaacttgattgatcgttttacagaaacttacgatgctggattacaaagacgaattgaacaatatatcactgctcaagtaactttacaaggattgagtaatccaagtggaagtttggcagatggctcaggactaggagagccaaagtttgaactaacccttaagccattcactgggaactggggtagaccacaaagagatggtcctgctttgagagcaatagccttaatcggctactcaaaatggttaatcaacaataactaccaatcaacagtttcaaatgttatctggccaattgttaggaatgatttgaactacgtggctcaatactggaaccagaccggtttcgacctttgggaagaggttaatggctcttcctttttcacagtggcaaatcagcatagagctttggttgaaggagctactttagcggccactctcggtcagtcaggttcagcttactcttctgtagctcctcaagtactttgttttctacagagattctgggtatcttctggtggttacgttgattctaacattaacacaaatgaagggcgtactggcaaagatgtgaatagcgtccttaccagcatccatacattcgatcctaatttgggttgtgatgccgggacgtttcaaccttgttctgacaaggctttgagcaatctgaaagtggttgttgatagtttcagaagcatctacggtgtaaacaagggtattccagctggtgctgccgtggctatcggcagatatgcagaagatgtctactataatggaaatccatggtacttggctacttttgccgcagcagaacagttgtacgacgccatctacgtttggaaaaagactggtagcattactgttacagctacatccttagcatttttccaagagttagtcccaggggtcacagcaggcacgtactcctcttctagttcaacctttaccaacatcataaacgctgtctccacctatgccgacggttttctatccgaggctgccaaatacgttcctgcagatggttctctagctgaacaatttgacagaaattcaggtactcctctgtcagcagtacacctcacatggagttacgcatcttttctgacagcagccgcgagaagagccggcatagttccaccaagttgggccaattcatcagcctctacaataccatctacatgctcaggcgcttctgttgtagggagttactctaggccaaccgctacttcattcccaccttcccaaactccaaaaccaggcgtaccttccggaacaccttataccccactcccttgcgctacaccaacttcagtcgcagtgacgtttcacgaattagtttccacacaatttggtcacacagtgaaagttgcaggaaatgccgctgctttgggcaattggtcaacttccgcagcggtagctttggacgctgttaactacagagataatcatccattgtggattggtacggtcaacctagaagctggtgacgtcgttgagtataagtatatcatagttggtcaagatggttccgtcacttgggagtcagatcctaatcatacttacactgttcctgccgtagcttgcgtcacacaagttgtgaaggaagatacttggcaatcttaa

SEQ ID NO: 2 - DNA, 63 bp, Zygosaccharomyces rouxii
  misc_feature (1)..(63): 63 nucleotide remnant of Zygosaccharomyces rouxii acetamidase gene
agctttgtttttcgtgaatctctacgtcagctactgtttatccgatggtactgtatcgcaacg

SEQ ID NO: 3 - protein, 383 aa, Nocardiopsis sp.
  misc_feature (1)..(383): Nocardiopsis sp. (NRRL 18262, Strain 10R)
MRPSPVASAIGTGALAFGLALAAAPGALAASGPLPQAPTPEAEAVSMKEALQRDLDLTPSEAESLLTAQDTAFEIDEAAAEAAGDAYGGSVFDTETLDLTVLVTDAAAVEAVEAAGAEAEVVDFGIEGLDEIVEDLNDAGTVPGVVGWYPDVEGDTVVLEVLEGSGADVDGLLAEAGVDASAVEVATTDEQPQVYADIIGGLAYTMGGRCSVGFAATNSAGQPGFVTAGHCGTVGTQVSIGNGRGVFERSVFPGNDAAFVRGTSNFTLTNLVSRYNSGGYATVSGSSAAPIGSSVCRSGSTTGWHCGTIQARGQSVSYPQGTVTNMTRTSVCAEPGDSGGSFISGTQAQGVTSGGSGNCRTGGTTYYQEVNPMINSWGVRLRT

SEQ ID NO: 4 - protein, 411 aa, Citrobacter braakii
  misc_feature (1)..(411): Citrobacter braakii phytase
EEQNGMKLERVVIVSRHGVRAPTKFTPIMKNVTPDQWPQWDVPLGWLTPRGGELVSELGQYQRLWFTSKGLLNNQTCPSPGQVAVIADTDQRTRKTGEAFLAGLAPKCQIQVHYQKDEEKNDPLFNPVKMGKCSFNTLQVKNAILERAGGNIELYTQRYQSSFRTLENVLNFSQSETCKTTEKSTKCTLPEALPSELKVTPDNVSLPGAWSLSSTLTEIFLLQEAQGMPQVAWGRITGEKEWRDLLSLHNAQFDLLQRTPEVARSRATPLLDMIDTALLTNGTTENRYGIKLPVSLLFIAGHDTNLANLSGALDLNWSLPGQPDNTPPGGELVFEKWKRTSDNTDWVQVSFVYQTLRDMRDIQPLSLEKPAGKVDLKLIACEEKNSQGMCSLKSFSRLIKEIRVPECAVTE

SEQ ID NO: 5 - protein, 444 aa, Aspergillus niger
  misc_feature (1)..(444): Aspergillus niger phytase (DSM)
ASRNQSSCDTVDQGYQCFSETSHLWGQYAPFFSLANESVISPEVPAGCRVTFAQVLSRHGARYPTDSKGKKYSALIEEIQQNATTFDGKYAFLKTYNYSLGADDLTPFGEQELVNSGIKFYQRYESLTRNIVPFIRSSGSSRVIASGKKFIEGFQSTKLKDPRAQPGQSSPKIDVVISEASSSNNTLDPGTCTVFEDSELADTVEANFTATFVPSIRQRLENDLSGVTLTDTEVTYLMDMCSFDTISTSTVDTKLSPFCDLFTHDEWINYDYLQSLKKYYGHGAGNPLGPTQGVGYANELIARLTHSPVHDDTSSNHTLDSSPATFPLNSTLYADFSHDNGIISILFALGLYNGTKPLSTTTVENITQTDGFSSAWTVPFASRLYVEMMQCQAEQEPLVRVLVNDRVVPLHGCPVDALGRCTRDSFVRGLSFARSGGDWAECFA

SEQ ID NO: 6 - protein, 450 aa, Rhizomucor pusillus
  misc_feature (1)..(450): Rhizomucor pusillus alpha-amylase
SPLPQQQRYGKRATSDDWKSKAIYQLLTDRFGRADDSTSNCSNLSNYCGGTYEGITKHLDYISGMGFDAIWISPIPKNSDGGYHGYWATDFYQLNSNFGDESQLKALIQAAHERDMYVMLDVVANHAGPTSNGYSGYTFGDASLYHPKCTIDYNDQTSIEQCWVADELPDIDTENSDNVAILNDIVSGWVGNYSFDGIRIDTVKHIRKDFWTGYAEAAGVFATGEVFNGDPAYVGPYQKYLPSLINYPMYYALNDVFVSKSKGFSRISEMLGSNRNAFEDTSVLTTFVDNHDNPRFLNSQSDKALFKNALTYVLLGEGIPIVYYGSEQGFSGGADPANREVLWTTNYDTSSDLYQFIKTVNSVRMKSNKAVYMDIYVGDNAYAFKHGDALVVLNNYGSGSTNQVSFSVSGKFDSGASLMDIVSNITTTVSSDGTVTFNLKDGLPAIFTSA

SEQ ID NO: 7 - protein, 591 aa, Taleromyces emersonii
  misc_feature (1)..(591): Taleromyces emersonii gluco-amylase
ATGSLDSFLATETPIALQGVLNNIGPNGADVAGASAGIVVASPSRSDPNYFYSWTRDAALTAKYLVDAFNRGNKDLEQTIQQYISAQAKVQTISNPSGDLSTGGLGEPKFNVNETAFTGPWGRPQRDGPALRATALIAYANYLIDNGEASTADEIIWPIVQNDLSYITQYWNSSTFDLWEEVEGSSFFTTAVQHRALVEGNALATRLNHTCSNCVSQAPQVLCFLQSYWTGSYVLANFGGSGRSGKDVNSILGSIHTFDPAGGCDDSTFQPCSARALANHKVVTDSFRSIYAINSGIAEGSAVAVGRYPEDVYQGGNPWYLATAAAAEQLYDAIYQWKKIGSISITDVSLPFFQDIYPSAAVGTYNSGSTTFNDIISAVQTYGDGYLSIVEKYTPSDGSLTEQFSRTDGTPLSASALTWSYASLLTASARRQSVVPASWGESSASSVLAVCSATSATGPYSTATNTVWPSSGSGSSTTTSSAPCTTPTSVAVTFDEIVSTSYGETIYLAGSIPELGNWSTASAIPLRADAYTNSNPLWYVTVNLPPGTSFEYKFFKNQTDGTIVWEDDPNRSYTVPAYCGQTTAILDDSWQ

SEQ ID NO: 8 - protein, 561 aa, Trametes cingulata
  misc_feature (1)..(561): Trametes cingulata gluco-amylase
QSSAADAYVASESPIAKAGVLANIGPSGSKSNGAKASDTPASXIASPSTSNPNYLYTWTRDSSLVFKALIDQFTTGEDTSLRTLIDEFTSAEAILQQVPNPSGTVSTGGLGEPKFNIDETAFTDAWGRPQRDGPALRATAIITYANWLLDNKNTTYVTNTLWPIIKLDLDYVASNWNQSTFDLWEEINSSSFFTTAVQHRALREGATFANRIGQTSVVSGYTTQANNLLCFLQASYWNPTGGYITANTGGGRSGKDANTVLTSIHTFDPAAGCDAVTFQPCSDKALSNLKVYVDAFRSIYSINSGIASNAAVATGRYPEDSYMGGNPWYLTTSAVAEQLYDALIVWNKLGALNVTSTSLPFFQQFSSGVTVGTYASSSSTFKTLTSAIKTFADGFLAVNAKYTPSNGGLAEQYSRSNGSPVSAVDLTWSYAAALTSFAARSGKTYASWGAAGLTVPTTCSGSGGAGTVAVTFNVQATTVFGENIYITGSVPALQNWSPDNALILSAANYPTWSITVNLPASTTIEYKYIRKFNGAVTWESDPNNSITTPASGTFTQNDTWR

SEQ ID NO: 9 - protein, 619 aa, Aspergillus kawachii
  misc_feature (1)..(619): Aspergillus kawachii alpha-amylase
LSAAEWRTQSIYFLLTDRFGRTDNSTTATCNTGDQIYCGGSWQGIINHLDYIQGMGFTAIWISPITEQLPQDTSDGEAYHGYWQQKIYNVNSNFGTADDLKSLSDALHARGMYLMVDVVPNHMGYAGNGNDVDYSVFDPFDSSSYFHPYCLITDWDNLTMVQDCWEGDTIVSLPDLNTTETAVRTIWYDWVADLVSNYSVDGLRIDSVEEVEPDFFPGYQEAAGVYCVGEVDNGNPALDCPYQKYLDGVLNYPIYWQLLYAFESSSGSISNLYNMIKSVASDCSDPTLLGNFIENHDNPRFASYTSDYSQAKNVLSYIFLSDGIPIVYAGEEQHYSGGDVPYNREATWLSGYDTSAELYTWIATTNAIRKLAISADSDYITYANDPIYTDSNTIAMRKGTSGSQIITVLSNKGSSGSSYTLTLSGSGYTSGTKLIEAYTCTSVTVDSNGDIPVPMASGLPRVLLPASVVDSSSLCGGSGNTTTTTTAATSTSKATTSSSSSSAAATTSSSCTATSTTLPITFEELVTTTYGEEVYLSGSISQLGEWDTSDAVKLSADDYTSSNPEWSVTVSLPVGTTFEYKFIKVDEGGSVTWESDPNREYTVPECGSGSGETVVDTWR

SEQ ID NO: 10 - protein, 634 aa, Humicola grisea
  misc_feature (1)..(634): Humicola grisea gluco-amylase
MHTFSKLLVLGSAVQSALGRPHGSSRLQERAAVDTFINTEKPIAWNKLLANIGPNGKAAPGAAAGVVIASPSRTDPPYFFTWTRDAALVLTGIIESLGHNYNTTLQTVIQNYVASQAKLQQVSNPSGTFADGSGLGEAKFNVDLTAFTGEWGRPQRDGPPLRAIALIQYAKWLIANGYKSTAKSVVWPVVKNDLAYTAQYWNETGFDLWEEVPGSSFFTIASSHRALTEGAYLAAQLDTECRACTTVAPQVLCFQQAFWNSKGNYVVSNINGGEYRSGKDANSILASIHNFDPEAGCDNLTFQPCSERALANHKAYVDSFRNLYAINKGIAQGKAVAVGRYSEDVYYNGNPWYLANFAAAEQLYDAIYVWNKQGSITVTSVSLPFFRDLVSSVSTGTYSKSSSTFTNIVNAVKAYADGFIEVAAKYTPSNGALAEQYDRNTGKPDSAADLTWSYSAFLSAIDRRAGLVPPSWRASVAKSQLPSTCSRIEVAGTYVAATSTSFPSKQTPNPSAAPSPSPYPTACADASEVYVTFNERVSTAWGETIKVVGNVPALGNWDTSKAVTLSASGYKSNDPLWSITVPIKATGSAVQYKYIKVGTNGKITWESDPNRSITLQTASSAGKCAAQTVNDSWR

SEQ ID NO: 11 - protein, 492 aa, Saccharomycopsis fibuligera
  misc_feature (1)..(492): Saccharomycopsis fibuligera gluco-amylase AE8
AYPSFEAYSNYKVDRTDLETFLDKQKDVSLYYLLQNIAYPEGQFNDGVPGTVIASPSTSNPDYYYQWTRDSAITFLTVLSELEDNNFNTTLAKAVEYYINTSYNLQRTSNPSGSFDDENHKGLGEPKFNTDGSAYTGAWGRPQNDGPALRAYAISRYLNDVNSLNKGKLVLTDSGDINFSSTEDIYKNIIKPDLEYVIGYWDSTGFDLWEENQGRHFFTSLVQQKALAYAVDIAKSFDDGDFANTLSSTASTLESYLSGSDGGFVNTDVNHIVENPDLLQQNSRQGLDSATYIGPLLTHDIGESSSTPFDVDNEYVLQSYYLLLEDNKDRYSVNSAYSAGAAIGRYPEDVYNGDGSSEGNPWFLATAYAAQVPYKLVYDAKSASNDITINKINYDFFNKYIVDLSTINSGYQSSDSVTIKSGSDEFNTVADNLVTFGDSFLQVILDHINDDGSLNEQLNRNTGYSTSAYSLTWSSGALLEAIRLRNKVKALA

SEQ ID NO: 12 - protein, 484 aa, Aspergillus niger
  misc_feature (1)..(484): Aspergillus niger alpha-amylase
LSAAEWRTQSIYFLLTDRFGRTDNSTTATCDTGDQIYCGGSWQGIINHLDYIQGMGFTAIWISPITEQLPQDTADGEAYHGYWQQKIYDVNSNFGTADDLKSLSDALHARGMYLMVDVVPNHMGYAGNGNDVDYSVFDPFDSSSYFHPYCLITDWDNLTMVQDCWEGDTIVSLPDLNTTETAVRTIWYDWVADLVSNYSVDGLRIDSVLEVEPDFFPGYQEAAGVYCVGEVDNGNPALDCPYQKVLDGVLNYPIYWQLLYAFESSSGSISNLYNMIKSVASDCSDPTLLGNFIENHDNPRFASYTSDYSQAKNVLSYIFLSDGIPIVYAGEEQHYSGGKVPYNREATWLSGYDTSAELYTWIATTNAIRKLAISADSAYITYANDAFYTDSNTIAMRKGTSGSQVITVLSNKGSSGSSYTLTLSGSGYTSGTKLIEAYTCTSVTVDSSGDIPVPMASGLPRVLLPASVVDSSSLCGGSGRLYVE

SEQ ID NO: 13 - protein, 1054 aa, Trichoderma reesei
  misc_feature (1)..(1054): Trichoderma reesei trehalase
TLVDRVTKCLSRHDGSDAESHFSKNVYKTDFAGVTWDEDNWLLSTTQLKQGAFEARGSVANGYLGINVASVGPFFEVDTEEDGDVISGWPLFSRRQSFATVAGFWDAQPQMNGTNFPWLSQYGSDTAISGIPHWSGLVLDLGGGTYLDATVSNKTISHFRSTYDYKAGVLSWSYKWTPKGNKGSFDISYRLFANKLHVNQAVVDMQVTASKNVQASIVNVLDGFAAVRTDFVESGEDGSAIFAAVRPNGVANVTAYVYADITGSGGVNLSSRKIVHNKPYVHANASSIAQAVPVKFAAGRTVRVTKFVGAASSDAFKNPKQVAKKAAAAGLSNGYTKSLKAHVEEWATVMPESSVDSFADPKTGKLPADSHIVDSAIIAVTNTYYLLQNTVGKNGIKAVDGAPVNVDSISVGGLTSDSYAGQIFWDADLWMQPGLVAAHPEAAERITNYRLAYGQAKENVKTAYAGSQNETFFSASAAVFPWTSGRYGNCTATGPCWDYEYHLNGDIGISLVNQWVVNGDTKDFEKNLFPVYDSVAQLYGNLLRPNKTSWTLTNMTDPDEYANHVDAGGYTMPLIAETLQKANSFRQQFGIEQNKTWNDMASNVLVLRENGVTLEFTAMNGTAVVKQADVIMLTYPLSYGTNYSAQDALNDLDYYANKQSPDGPAMTYAFFSIVANEISPSGCSAYTYAQNAFKPYVRAPFYQISEQLIDDASVNGGTHPAYPFLTGHGGAHQVVLFGYLGLRLVPDDVIHIEPNLPPQIPYLRYRTFYWRGWPISAWSNYTHTTLSRAAGVAALEGADQRFARKPITIHAGPEQDPTAYRLPVKGSVVIPNKQIGSQQTYAGNLVQCHAASSPNDYVPGQFPIAAVDGATSTKWQPASADKVSSITVSLDKEDVGSLVSGFHFDWAQAPPVNATVIFHDEALADPATALASAHKHNSKYTTVTSLTNIELSDPYVSTKDLNAIAIPIGNTTNVTLSHPVAASRYASLLIVGNQGLDPVDVKAKNGTGATVAEWAIFGHGKEHSGKPSSHSKRRLNVRTAATLSNPRSFMRRRL

SEQ ID NO: 14 - protein, 928 aa, Bacillus deramificans
  misc_feature (1)..(928): Bacillus deramificans pullulanase
DGNTTTIIVHYFRPAGDYQPWSLWMWPKDGGGAEYDFNQPADSLGAVASADIPGNPSQVGIIVRTQDWTKDVSADRYIDLSKGNEVWLVEGNSQIFYSEKDAEDAAKPAVSNAYLDASNQVLVKLSQPLTLGEGASGFTVHDDTANKDIPVTSVKDASLGQDVTAVLAGTFQHIFGGSDWAPDNHSTLLKKVTNNLYQFSGDLPEGNYQYKVALNDSWNNPSYPSDNINLTVPAGGAHVTFSYIPSTHAVYDTINNPNADLQVESGVKTDLVTVTLGEDPDVSHTLSIQTDGYQAKQVIPRNVLNSSQYYYSGDDLGNTYTQKATTFKVWAPTSTQVNVLLYDSATGSVTKIVPMTASGHGVWEATVNQNLENWYYMYEVTGQGSTRTAVDPYATAIAPNGTRGMIVDLAKTDPAGWNSDKHITPKNIEDEVIYEMDVRDFSIDPNSGMKNKGKYLALTEKGTKGPDNVKTGIDSLKQLGITHVQLMPVFASNSVDETDPTQDNWGYDPRNYDVPEGQYATNANGNARIKEFKEMVLSLHREHIGVNMDVVYNHTFATQISDFDKIVPEYYYRTDDAGNYTNGSGTGNEIAAERPMVQKFIIDSLKYWVNEYHIDGFRFDLMALLGKDTMSKAASELHAINPGIALYGEPWTGGTSALPDDQLLTKGAQKGMGVAVFNDNLRNALDGNVFDSSAQGFATGATGLTDAIKNGVEGSINDFTSSPGETINYVTSHDNYTLWDKIALSNPNDSEADRIKMDELAQAVVMTSQGVPFMQGGEEMLRTKGGNDNSYNAGDAVNEFDWSRKAQYPDVFNYYSGLIHLRLDHPAFRMTTANEINSHLQFLNSPENTVAYELTDHVNKDKWGNIIVVYNPNKTVATINLPSGKWAINATSGKVGESTLGQAEGSVQVPGISMMILHQEVSPDHGKK

SEQ ID NO: 15 - protein, 413 aa, Buttiauxella sp.
  misc_feature (1)..(413): Buttiauxella sp. Phytase
NDTPASGYQVEKVVILSRHGVRAPTKMTQTMRDVTPNTWPEWPVKLGYITPRGEHLISLMGGFYRQKFQQQGILSQGSCPTPNSIYVWADVDQRTLKTGEAFLAGLAPQCGLTIHHQQNLEKADPLFHPVKAGTCSMDKTQVQQAVEKEAQTPIDNLNQHYIPFLALMNTTLNFSTSAWCQKHSADKSCDLGLSMPSKLSIKDNGNKVALDGAIGLSSTLAEIFLLEYAQGMPQAAWGNIHSEQEWASLLKLHNVQFDLMARTPYIARHNGTPLLQAISNALNPNATESKLPDISPDNKILFIAGHDTNIANIAGMLNMRWTLPGQPDNTPPGGALVFERLADKSGKQYVSVSMVYQTLEQLRSQTPLSLNQPAGSVQLKIPGCNDQTAEGYCPLSTFTRVVSQSVEPGCQLQ

SEQ ID NO: 16 - protein, 387 aa, Trichoderma reesei
  misc_feature (1)..(387): Trichoderma reesei protease
LPTEGQKTASVEVQYNKNYVPHGPTALFKAKRKYGAPISDNLKSLVAARQAKQALAKRQTGSAPNHPSDSADSEYITSVSIGTPAQVLPLDFDTGSSDLWVFSSETPKSSATGHAIYTPSKSSTSKKVSGASWSISYGDGSSSSGDVYTDKVTIGGFSVNTQGVESATRVSTEFVQDTVISGLVGLAFDSGNQVRPHPQKTWFSNAASSLAEPLFTADLRHGQNGSYNFGYIDTSVAKGPVAYTPVDNSQGFWEFTASGYSVGGGKLNRNSIDGIADTGTTLLLLDDNVVDAYYANVQSAQYDNQQEGVVFDCDEDLPSFSFGVGSSTITIPGDLLNLTPLEEGSSTCFGGLQSSSGIGINIFGDVALKAALVVFDLGNERLGWAQK

SEQ ID NO: 17 - protein, 636 aa, Aspergillus clavatus
  misc_feature (1)..(636): full-length Aspergillus clavatus alpha-amylase
MKLLALTTAFALLGKGVFGLTPAEWRGQSIYFLITDRFARTDGSTTAPCDLSQRAYCGGSWQGIIKQLDYIQGMGFTAIWITPITEQIPQDTAEGSAFHGYWQKDIYNVNSHFGTADDIRALSKALHDRGMYLMIDVVANHMGYNGPGASTDFSTFTPFNSASYFHSYCPINNYNDQSQVENCWLGDNTVALADLYTQHSDVRNIWYSWIKEIVGNYSADGLRIDTVKHVEKDFWTGYTQAAGVYTVGEVLDGDPAYTCPYQGYVDGVLNYPIYYPLLRAFESSSGSMGDLYNMINSVASDCKDPTVLGSFIENHDNPRFASYTKDMSQAKAVISYVILSDGIPIIYSGQEQHYSGGNDPYNREAIWLSGYSTTSELYKFIATTNKIRQLAISKDSSYLTSRNNPFYTDSNTIAMRKGSGGSQVITVLSNSGSNGGSYTLNLGNSGYSSGANLVEVYTCSSVTVGSDGKIPVPMASGLPRVLVPASWMSGSGLCGSSSTTTLVTATTTPTGSSSSTTLATAVTTPTGSCKTATTVPVVLEESVRTSYGENIFISGSIPQLGSWNPDKAVALSSSQYTSSNPLWAVTLDLPVGTSFEYKFLKKEQNGGVAWENDPNRSYTVPEACAGTSQKVDSSWR

SEQ ID NO: 18 - protein, 632 aa, Trichoderma reesi
  misc_feature (1)..(632): Full length Trichoderma reesi engineered glucoamylase exemplified in Example 1
MHVLSTAVLLGSVAVQKVLGRPGSSGLSDVTKRSVDDFISTETPIALNNLLCNVGPDGCRAFGTSAGAVIASPSTIDPDYYYMWTRDSALVFKNLIDRFTETYDAGLQRRIEQYITAQVTLQGLSNPSGSLADGSGLGEPKFELTLKPFTGNWGRPQRDGPALRAIALIGYSKWLINNNYQSTVSNVIWPIVRNDLNYVAQYWNQTGFDLWEEVNGSSFFTVANQHRALVEGATLAATLGQSGSAYSSVAPQVLCFLQRFWVSSGGYVDSNINTNEGRTGKDVNSVLTSIHTFDPNLGCDAGTFQPCSDKALSNLKVVVDSFRSIYGVNKGIPAGAAVAIGRYAEDVYYNGNPWYLATFAAAEQLYDAIYVWKKTGSITVTATSLAFFQELVPGVTAGTYSSSSSTFTNIINAVSTYADGFLSEAAKYVPADGSLAEQFDRNSGTPLSAVHLTWSYASFLTAAARRAGIVPPSWANSSASTIPSTCSGASVVGSYSRPTATSFPPSQTPKPGVPSGTPYTPLPCATPTSVAVTFHELVSTQFGHTVKVAGNAAALGNWSTSAAVALDAVNYRDNHPLWIGTVNLEAGDVVEYKYIIVGQDGSVTWESDPNHTYTVPAVACVTQVVKEDTWQS

SEQ ID NO: 19 - protein, 462 aa, Rhizopus oryzae
  misc_feature (1)..(462): Rhizopus oryzae alpha-amylase
MKSFLSLLCSVFLLPLVVQSVPVIKRASASDWENRVIYQLLTDRFAKSTDDTNGCNNLSDYCGGTFQGIINHLDYIAGMGFDAIWISPIPKNANGGYHGYWATDFSQINEHFGTADDLKKLVAAAHAKNMYVMLDVVANHAGIPSSGGDYSGYTFGQSSEYHTACDINYNSQTSIEQCWISGLPDINTEDSAIVSKLNSIVSGWVSDYGFDGLRIDTVKHIRKDFWDGYVSAAGVFATGEVLSGDVSYVSPYQQHVPSLLNYPLYYPVYDVFTKSRTMSRLSSGFSDIKNGNFKNIDVLVNFIDNHDQPRLLSKADQSLVKNALAYSFMVQGIPVLYYGTEQSFKGGNDPNNREVLWTTGYSTTSDMYKFVTTLVKARKGSNSTVNMGIAQTDNVYVFQRGGSLVVVNNYGQGSTNTITVKAGSFSNGDTLTDVFSNKSVTVQNNQITFQLQNGNPAIFQKK

(19) United States
(12) Patent Application Publication
(10) Pub. No.: US 2016/0264927 A1
(43) Pub. Date: Sep. 15, 2016

(54) Large Scale Genetically Engineered Active Dry Yeast

(71) Applicant: DANISCO US INC., Palo Alto, CA (US)

(72) Inventors: Andrei Miasnikov, Union City, CA (US); Douglas A. Dale, Pacifica, CA (US); Douglas Ko, Milpitas, CA (US); George England, Redwood City, CA (US); Wyatt C. Smith, Tiburon, CA (US); Beth Fryksdale, San Jose, CA (US); Matthew T. Reboli, Sunnyvale, CA (US)

(73) Assignee: Danisco US Inc., Palo Alto, CA (US)

(21) Appl. No.: 15/031,833

(22) PCT Filed: Oct. 27, 2014
(86) PCT No.: PCT/US2014/062327

§ 371 Date: Apr. 25, 2016

Related U.S. Application Data
(60) Provisional application No. 61/896,525, filed on Oct. 28, 2013.
(60) Provisional application No. 61/896,869, filed on Oct. 29, 2013.

(51) Int. Cl. C12N 1/18; C12N 9/34; C12P 7/06

(52) U.S. Cl. CPC C12N 1/18; C12P 7/06; C12N 9/2428; C12Y 302/01003

(57) ABSTRACT

The present teachings provide large scale genetically engineered yeast in active dry form.

### CROSS REFERENCE TO RELATED APPLICATIONS

This application claims benefit of priority from U.S. Provisional Patent Nos. U.S. Ser. No. 61/896,525, filed 28 Oct. 2013 and U.S. Ser. No. 61/896,869, filed 29 Oct. 2013, the contents of which are incorporated herein by reference in their entirety.

### FIELD OF THE INVENTION

This disclosure is directed towards improved microbes for use in fermentation processes, especially for example biofuel generation.

### BACKGROUND

Interest is growing in the use of sustainable and economical biological processes for generating materials of interest. Biological processes hold the promise of renewably using energy from the sun to make such materials. For example, energy from the sun can be stored in plant biomolecules such as the polysaccharides starch and cellulose. By fermentation of the simple sugars arising from breakdown of these polysaccharides, microbes can transfer the sun's energy into molecules of commercial interest to humans, including ethanol. Historically, large-scale polysaccharide breakdown has been accomplished by heat and chemicals, but in the past decades industrially produced starch hydrolytic enzymes have been employed to facilitate this process.

The tools of recombinant DNA technology arising in the 1980's have enabled the creation of transgenic organisms capable of expressing high levels of starch hydrolysis enzymes. In routine use today are alpha amylases, glucoamylases, and pullulanases, produced by recombinant microbes at the scale of tanker trucks per day. However, making biomolecules of interest by this process is lengthy and inherently inefficient. For example, energy is first transferred from the sun to plant polysaccharides, then from these plant polysaccharides to microbes that make starch hydrolysis enzymes, and then the enzymes thus produced are used to facilitate breakdown of additional plant polysaccharides used by yet another microbe to eventually form ethanol. Accordingly, using the same microbe that produces the material of interest to also produce the starch hydrolysis enzyme offers the opportunity for more efficient resource utilization (see for example, U.S. Pat. No. 5,422,267).

Such approaches have recently come to commercial fruition in the form of a glucoamylase-expressing yeast in the fuel ethanol industry. These approaches promise to reduce the use of expensive exogenously added enzymes. However, in this infant industry setting many unmet needs exist. One large need resides in the production and formulation of easily transportable and easily useable highly active genetically engineered microbes. The present invention advances this work.

### SUMMARY

The present teachings provide novel genetically engineered yeast strains. In some embodiments, the genetically yeast strains are grown to produce large scale active dry yeast at previously unknown levels. In some embodiments, the yeast of the present teachings is used to ferment ethanol, and to reduce the use of exogenously added enzymes such as glucoamylases.

### BRIEF DESCRIPTION OF THE FIGURES

FIG. 1 depicts some illustrative data according to some embodiments of the present teachings.

### DETAILED DESCRIPTION

The practice of the present teachings will employ, unless otherwise indicated, conventional techniques of molecular biology (including recombinant techniques), microbiology, cell biology, biochemistry, and animal feed pelleting, which are within the skill of the art. Such techniques are explained fully in the literature, for example, Molecular Cloning: A Laboratory Manual, second edition (Sambrook et al., 1989); Oligonucleotide Synthesis (M. J. Gait, ed., 1984; Current Protocols in Molecular Biology (F. M. Ausubel et al., eds., 1994); PCR: The Polymerase Chain Reaction (Mullis et al., eds., 1994); Gene Transfer and Expression: A Laboratory Manual (Kriegler, 1990), and The Alcohol Textbook (Ingledew et al., eds., Fifth Edition, 2009).

Unless defined otherwise herein, all technical and scientific terms used herein have the same meaning as commonly understood by one of ordinary skill in the art to which the present teachings belong. Singleton, et al., Dictionary of Microbiology and Molecular Biology, second ed., John Wiley and Sons, New York (1994), and Hale & Markham, The Harper Collins Dictionary of Biology, Harper Perennial, N.Y. (1991) provide one of skill with a general dictionary of many of the terms used in this invention. Any methods and materials similar or equivalent to those described herein can be used in the practice or testing of the present teachings.

Numeric ranges provided herein are inclusive of the numbers defining the range.

### DEFINITIONS

As used herein, the term “active dry form” refers to a yeast made according to the present teachings in which the resulting product has at least 1×10⁸, 1×10⁹, 1×10¹⁰, or 2×10¹⁰ total yeast cells per gram, with at least 50%, 60%, 70%, or 75% viable cells, and has a moisture content of 3-10%, 4-9%, or 5-8%. In some embodiments, the active dry form comprises at least 2×10¹⁰ total yeast cells per gram, at least 75% viable cells, and 5-8% moisture content. Active dry form is used interchangeably herein with “ADY product”.

As used herein, the determination of “total yeast cells per gram” and the determination of “viable cells” are made according to the following procedure. ADY sample is diluted in a Butterfield's Buffer (3M) and incubated, with frequent vortexing to keep in suspension, in a 35° C. water bath and is analyzed within 2 hours using a fluorescence microscope (Nucleocounter YC-100 from Chemometec) to measure total count and number of viable yeast cells. The rehydrated ADY sample is treated with a fluorescence marker (propidium iodide, PI), to which non-viable yeast are permeable. To determine total count, the sample is treated with lysis agent to render all yeast cells non-viable, and then treated with PI to determine total count.

As used herein, the term “at least one additional recombinant gene” refers to a nucleic acid encoding a protein that is integrated into the genome of the yeast, in addition to the at least one recombinant gene for hydrolyzing starch. Examples are numerous as will be appreciated by one of skill in the art, and include any of the genes mentioned herein.

As used herein, the term “genetically engineered yeast” refers to the targeted modification of at least one nucleotide into a nucleotide sequence resulting in a sequence that does not naturally occur. Such a genetic engineering can be the targeted modification of an endogenous wild type gene, the targeted modification of an endogenous wild type non-coding region, and/or through the insertion of a different organism's gene or non-coding sequence (such different organism's gene or non-coding region itself optionally having been the subject of targeted modification) into the yeast (the use of such a different organism's genetic material aka “recombinant”). Mere genetic changes in a yeast that arise through mutagenesis and screening are not considered by themselves in the present invention to constitute a “genetically engineered yeast”. Examples of genes that can constitute a genetically engineered yeast are numerous, and include any of phytases, xylanases, β-glucanases, phosphatases, proteases, amylases (alpha or beta or glucoamylases), pullulanases, isoamylases, cellulases, trehalases, lipases, pectinases, polyesterases, cutinases, oxidases, transferases, reductases, hemicellulases, mannanases, esterases, isomerases, pectinases, lactases, peroxidases, laccases, and redox enzymes. Indeed, any enzyme can be used according to the present teachings, and a nonlimiting examples include a xylanase from Trichoderma reesei and a variant xylanase from Trichoderma reesei, both available from DuPont Industrial Biosciences or the inherently thermostable xylanase described in EP1222256B1, as well as other xylanases from Aspergillus niger, Aspergillus kawachii, Aspergillus tubigensis, Bacillus circulans, Bacillus pumilus, Bacillus subtilis, Neocallimastix patriciarum, Penicillium species, Streptomyces lividans, Streptomyces thermoviolaceus, Thermomonospora fusca, Trichoderma harzianurn, Trichoderma reesei, Trichoderma viride. Additional enzymes include phytases, such as for example Finase L®, a phytase from Aspergillus sp., available from AB Enzymes, Darmstadt, Germany; Phyzyme™ XP, a phytase from E. Coli, available from DuPont Nutrition and Health, and other phytases from, for example, the following organisms: Trichoderma, Penicillium, Fusarium, Buttiauxella, Citrobacter, Enterobacter, Penicillium, Humicola, Bacillus, and Peniophora. An example of a cellulase is Multifect® BGL, a cellulase (beta glucanase), available from DuPont Industrial Biosciences and other cellulases from species such as Aspergillus, Trichoderma, Penicillium, Humicola, Bacillus, Cellulomonas, Penicillium, Thermomonospore, Clostridium, and Hypocrea. The cellulases and endoglucanases described in US20060193897A1 also may be used. Amylases may be, for example, from species such as Aspergillus, Trichoderma, Penicillium, Bacillus, for instance, B. subtilis, B. stearothermophilus, B. lentus, B. licheniformis, B. coagulans, and B. amyloliquefaciens. Suitable fungal amylases are derived from Aspergillus, such as A. oryzae and A. niger. Proteases may be from Bacillus amyloliquefaciens, Bacillus lentus, Bacillus subtilis, Bacillus licheniformis, and Aspergillus and Trichoderma species. In some embodiments, any of the enzymes in the sequence listing may be used, either alone, or in combination with themselves, or others. In some embodiments, the present teachings provide a genetically modified yeast containing at least one nucleic acid encoding at least one of the amino acid sequences present in the sequence listing. In some embodiments, the present teachings provide a genetically modified yeast comprising at least one nucleic acid encoding at least one of the amino acid sequences present in the sequence listing, at least one nucleic acid encoding an amino acid 99%, 98%, 97%, 95%, 90%, 85%, or 80% identical to at least one of the amino acid sequences present in the sequence listing. One of skill in the art will appreciate that various engineering efforts have produced improved enzymes with properties of interest, any of which can be included in a genetically engineered yeast according to the present teachings. For example, in the context of amylases, various swapping and mutatation of starch binding modules and/or carbohydrate modules (cellulose, starch, or otherwise) have generated enzymes of interest that could be placed into the genetically engineered yeast of the present teachings (see for example, U.S. Pat. No. 8,076,109, and EP1687419B1, as well as Machovic, Cell. Mol. Life Sc. 63 (2006) 2710-2724, and Latorre-Garcia, J. biotech, 2005 (3, 019) 167-176). As another example, the Rhizomucor pusillus alpha-amylase in the sequence listing can be combined with any CBM. Also, the present teachings can employ any of the enzymes disclosed in PCT/US2009/036283, Moraes et. al., Appl Microbiol Biotechnol (1995) 43:1067-1076, and Li et. al., Protein Expression and Purification 79 (2011) 142-148. In certain embodiments, the microorganism may be genetically modified to produce butanol. It will also be appreciated that in some embodiments the production of butanol by a microorganism, is disclosed, for example, in U.S. Pat. Nos. 7,851,188; 7,993,889; 8,178,328; and 8,206,970; and U.S. Patent Application Publication Nos. 2007/0292927; 2008/0182308; 2008/0274525; 2009/0305363; 2009/0305370; 2011/0250610; 2011/0313206; 2011/0111472; 2012/0258873; and 2013/0071898, the entire contents of each are herein incorporated by reference. In certain embodiments, the microorganism is genetically modified to comprise a butanol biosynthetic pathway or a biosynthetic pathway for a butanol isomer, such as 1-butanol, 2-butanol, or isobutanol. In certain embodiments, at least one, at least two, at least three, at least four, or at least five polypeptides catalyzing substrate to product conversions in the butanol biosynthetic pathway are encoded by heterologous polynucleotides in the microorganism. In certain embodiments, all the polypeptides catalyzing substrate to product conversions of the butanol biosynthetic pathway are encoded by heterologous polynucleotides in the microorganism. It will be appreciated that microorganisms comprising a butanol biosynthetic pathway may further comprise one or more additional genetic modifications as disclosed in U.S. Patent Application Publication No. 2013/0071898, which is herein incorporated by reference in its entirety. Biosynthetic pathways for the production of isobutanol that may be used include those as described by Donaldson et al. in U.S. Pat. No. 7,851,188; U.S. Pat. No. 7,993,388; and International Publication No. WO 2007/050671, which are incorporated herein by reference. Biosynthetic pathways for the production of 1-butanol that may be used include those described in U.S. Patent Application Publication No. 2008/0182308 and WO2007/041269, which are incorporated herein by reference. Biosynthetic pathways for the production of 2-butanol that may be used include those described by Donaldson et al. in U.S. Pat. No. 8,206,970; U.S. Patent Application Publication Nos. 2007/0292927 and 2009/0155870; International Publication Nos. WO 2007/130518 and WO 2007/130521, all of which are incorporated herein by reference. In some embodiments, the present teachings also contemplate the incorporation of a trehalase into a yeast to generate the genetically modified organism, either alone or with other enzymes of interest. Exemplary trehalases can be found in U.S. Pat. No. 5,312,909, EPO451896B1, and WO2009121058A9. Additional examples of enzymes, including starch hydrolysis enzymes, that can placed into the genetically engineered yeast of the present teachings include those described in U.S. Pat. No. 7,867,743, U.S. Pat. No. 8,512,986, U.S. Pat. No. 7,060,468, U.S. Pat. No. 6,620,924, U.S. Pat. No. 6,255,084, WO 2007134207, U.S. Pat. No. 7,332,319, U.S. Pat. No. 7,262,041, WO 2009037279, U.S. Pat. No. 7,968,691, and U.S. Pat. No. 7,541,026, all of which are incorporated by reference in their entirety.

As used herein, the term “an additional yeast species” refers to the existence of another yeast, or more, that is grown to scale along with the genetically engineered yeast and comprises the active dry yeast formulation. Such an additional yeast can itself be a genetically engineered yeast, but need not be.

As used herein, the term “Percent sequence identity” means that a variant has at least a certain percentage of amino acid residues identical to a reference sequence when aligned using the CLUSTAL W algorithm with default parameters. See Thompson et al. (1994) Nucleic Acids Res. 22:4673-4680. Default parameters for the CLUSTAL W algorithm are:
Gap opening penalty: 10.0
Gap extension penalty: 0.05
Protein weight matrix: BLOSUM series
DNA weight matrix: IUB
Delay divergent sequences %: 40
Gap separation distance: 8
DNA transitions weight: 0.50
List hydrophilic residues: GPSNDQEKR
Use negative matrix: OFF
Toggle Residue specific penalties: ON
Toggle hydrophilic penalties: ON
Toggle end gap separation penalty OFF.

Deletions are counted as non-identical residues, compared to a reference sequence. Deletions occurring at either terminus are included. For example, a variant with five amino acid deletions of the C-terminus of a mature 617 residue polypeptide would have a percent sequence identity of 99% (612/617 identical residues×100, rounded to the nearest whole number) relative to the mature polypeptide. Such a variant would be encompassed by a variant having “at least 99% sequence identity” to a mature polypeptide.

### EXEMPLARY EMBODIMENTS

In some embodiments, the present teachings provide a yeast formulation comprising at least one kilogram of a genetically engineered yeast in active dry form. In some embodiments, the yeast formulation comprises at least one recombinant gene for hydrolyzing starch, for example, SEQ ID NO: 1, or any glucoamylase provide in U.S. Pat. No. 7,494,685 and U.S. Pat. No. 7,413,887. In some embodiments, the genetically engineered yeast comprises at least one engineered nucleotide change into an endogenous gene, for example a trehalase gene. In some embodiments, the yeast formulation comprises a recombinant glucoamylase. In some embodiments, the genetically engineered yeast comprises SEQ ID NO: 1 or an enzyme 80%, 85%, 90%, 95%, or 99% identical thereto. In some embodiments, a genetically modified yeast is provided that contains at least one additional recombinant gene, wherein the at least one additional recombinant gene encodes an alpha amylase, a glucoamylase, a cutinase, trehalase, or any of the other enzymes recited herein, or known to one of ordinary skill in the art. In some embodiments, the yeast of the present teachings comprises SEQ ID NO: 2. In some embodiments, the species is Saccharomyces cerevisiae. In some embodiments, the yeast formulation comprises an additional yeast species.

In some embodiments, the present teachings provide a method of making at least one kilogram of genetically engineered yeast in active dry form comprising; growing a genetically modified yeast in a fermentation medium comprising at least 10,000 liters; recovering the yeast wherein no washing is performed; and, formulating an active dry form yeast, wherein the resulting active dry form yeast maintain equivalent viability compared to a control group in which washing was performed. In some embodiments, the formulating comprises fluid bed drying.

In some embodiments, the present teachings provide a method of making a desired biochemical comprising including the yeast provided by the present teachings in a fermentation process with a feedstock, wherein the desired biochemical is selected from the group consisting of ethanol, butanol, etc. arabinitol, n-butanol, isobutanol, ethanol, glycerol, methanol, ethylene glycol, 1,3-propanediol [propylene glycol], butanediol, glycerin, sorbitol, and xylitol); an alkane (e.g., pentane, hexane, heptane, octane, nonane, decane, undecane, and dodecane), a cycloalkane (e.g., cyclopentane, cyclohexane, cycloheptane, and cyclooctane), an alkene (e.g. pentene, hexene, heptene, and octene); an amino acid (e.g., aspartic acid, glutamic acid, glycine, lysine, serine, tryptophan, and threonine); a gas (e.g., methane, hydrogen (H2), carbon dioxide (CO₂), and carbon monoxide (CO)); isoprene, isoprenoid, sesquiterpene; a ketone (e.g., acetone); an aldehyde (e.g., acetaldehyde, butryladehyde); an organic acid (e.g., acetic acid, acetonic acid, adipic acid, ascorbic acid, citric acid, 2,5-diketo-Dgluconic acid, formic acid, fumaric acid, glucaric acid, gluconic acid, glucuronic acid, glutaric acid, 3-hydroxypropionic acid, itaconic acid, lactic acid, malic acid, malonic acid, oxalic acid, oxaloacetic acid, propionic acid, succinic acid, and xylonic acid); 1-3 propane diol, and polyketide. It will be appreciate that the feedstock is not a limitation of the present teachings, and can include for example, glucose, glucose syrups, sucrose, sucrose syrups, liquifact from starch, granular starch, and various cellulosic feedstocks appropriately treated to liberate fermentable sugars. In some embodiments, the feedstock is selected from the group consisting of glucose, liquefied starch, granular starch, or cellulose.

In some embodiments, the present teachings provide a Saccharomyces cerevisiae yeast comprising SEQ ID NO: 1 or a sequence 90%, 95%, 98%, or 99% identical to it. In some embodiments, the Saccharomyces cerevisiae yeast further comprises SEQ ID NO: 2.

In some embodiments, the present teachings provide a yeast comprising a nucleic acid encoding any of the sequences provided in the sequence listing. In some embodiments, such a yeast is present in at least 1 kg, 5 kg, or 10 kg active dry form as provided by the present teachings, and may contain at least 1×10⁸, 1×10⁹, 1×10¹⁰, or 2×10¹⁰ total yeast cells per gram, with at least 50%, 60%, 70%, or 75% viable cells, and comprise a moisture content of 3-10%, 4-9%, or 5-8%. In some embodiments, the active dry form comprises a yeast with a nucleic acid encoding any of the amino acid sequences of the sequence listing, and, at least 2×10¹⁰ total yeast cells per gram, at least 75% viable cells, and 5-8% moisture content.

In some embodiments, the present teachings provide at least 1 kilogram of active dry yeast, wherein the active dry yeast comprises a nucleic acid encoding at least one of the sequences in the sequence listing, or encodes an amino acid sequence 99%, 98%, 97%, 95%, 90%, 85%, or 80% identical to one of the sequences in the sequence listing, and further comprises a moisture content of 4-9%, at least 1×10¹⁰ total yeast cells per gram, and at least 75% viable cells.

In some embodiments, the present teachings provide at least 1 kilogram of active dry yeast, wherein the active dry yeast comprises a nucleic acid encoding the Taleromyces emersonii gluco-amylase of Exhibit 1, or encodes an amino acid sequence 99%, 98%, 97%, 95%, 90%, 85%, or 80% identical to the Taleromyces emersonii gluco-amylase in the sequence listing, and further comprises a moisture content of 4-9%, at least 1×10¹⁰ total yeast cells per gram, and at least 75% viable cells. In some embodiments, the present teachings provide at least 1 kilogram of active dry yeast, wherein the active dry yeast comprises a nucleic acid encoding the Taleromyces emersonii gluco-amylase of Exhibit 1, or encodes an amino acid sequence 99%, 98%, 97%, 95%, 90%, 85%, or 80% identical to the Taleromyces emersonii gluco-amylase in the sequence listing, and further comprises a moisture content of 5-8%, at least 2×10¹⁰ total yeast cells per gram, and at least 75% viable cells. In some embodiments, the present teachings provide at least 1 kilogram of active dry yeast, wherein the active dry yeast comprises a nucleic acid encoding the Taleromyces emersonii gluco-amylase of Exhibit 1, or encodes an amino acid sequence 98% identical to the Taleromyces emersonii gluco-amylase in the sequence listing, and further comprises a moisture content of 5-8%, at least 2×10¹⁰ total yeast cells per gram, and at least 75% viable cells.

In some embodiments, the present teachings provide at least 1 kilogram of active dry yeast, wherein the active dry yeast comprises a nucleic acid encoding the Trametes cingulata gluco-amylase of Exhibit 1, or encodes an amino acid sequence 99%, 98%, 97%, 95%, 90%, 85%, or 80% identical to the Trametes cingulata gluco-amylase in the sequence listing, and further comprises a moisture content of 4-9%, at least 1×10¹⁰ total yeast cells per gram, and at least 75% viable cells. In some embodiments, the present teachings provide at least 1 kilogram of active dry yeast, wherein the active dry yeast comprises a nucleic acid encoding the Trametes cingulata gluco-amylase of Exhibit 1, or encodes an amino acid sequence 99%, 98%, 97%, 95%, 90%, 85%, or 80% identical to the Trametes cingulata gluco-amylase in the sequence listing, and further comprises a moisture content of 5-8%, at least 2×10¹⁰ total yeast cells per gram, and at least 75% viable cells. In some embodiments, the present teachings provide at least 1 kilogram of active dry yeast, wherein the active dry yeast comprises a nucleic acid encoding the Trametes cingulata gluco-amylase of Exhibit 1, or encodes an amino acid sequence 98% identical to the Trametes cingulata gluco-amylase in the sequence listing, and further comprises a moisture content of 5-8%, at least 2×10¹⁰ total yeast cells per gram, and at least 75% viable cells.

In some embodiments, the present teachings provide at least 1 kilogram of active dry yeast, wherein the active dry yeast comprises a nucleic acid encoding the Humicola grisea gluco-amylase of Exhibit 1, or encodes an amino acid sequence 99%, 98%, 97%, 95%, 90%, 85%, or 80% identical to the Humicola grisea gluco-amylase in the sequence listing, and further comprises a moisture content of 4-9%, at least 1×10¹⁰ total yeast cells per gram, and at least 75% viable cells. In some embodiments, the present teachings provide at least 1 kilogram of active dry yeast, wherein the active dry yeast comprises a nucleic acid encoding the Humicola grisea gluco-amylase of Exhibit 1, or encodes an amino acid sequence 99%, 98%, 97%, 95%, 90%, 85%, or 80% identical to the Humicola grisea gluco-amylase in the sequence listing, and further comprises a moisture content of 5-8%, at least 2×10¹⁰ total yeast cells per gram, and at least 75% viable cells. In some embodiments, the present teachings provide at least 1 kilogram of active dry yeast, wherein the active dry yeast comprises a nucleic acid encoding the Humicola grisea gluco-amylase of Exhibit 1, or encodes an amino acid sequence 98% identical to the Humicola grisea gluco-amylase in the sequence listing, and further comprises a moisture content of 5-8%, at least 2×10¹⁰ total yeast cells per gram, and at least 75% viable cells.

In some embodiments, the present teachings provide at least 1 kilogram of active dry yeast, wherein the active dry yeast comprises a nucleic acid encoding a Thermoascus aurantiacus metalloprotease, or a molecule 99%, 98%, 97%, 95%, 90%, 85%, or 80% identical to a Thermoascus aurantiacus metalloprotease protease.

In some embodiments, the present teachings provide at least 1 kilogram of active dry yeast, wherein the active dry yeast comprises a nucleic acid encoding a Pyrococcus furiosis protease, or a molecule 99%, 98%, 97%, 95%, 90%, 85%, or 80% identical to a Pyrococcus furiosis protease.

### EXAMPLES

### Example 1

### Strain Engineering

The strain was constructed using genetic engineering techniques is such a way that no functional DNA except the expression cassette and (endogenous) URA3 marker gene were integrated into yeast genome. More specifically, a synthetic nucleotide sequence encoding a variant of the Trichoderma reseei glucoamylase gene was placed under control of native Saccharomyces cerevisiae FBA1 promoter and transcription terminator. The sequence of this Trichoderma reseii glucoamylase gene is shown as SEQ ID NO: 1.

SEQ ID NO: 1
ATGCTACTCCAAGCATTCCTTTTTCTGTTAGCAGGATTTGCTGCCAAAAT
CTCTGCTAGACCTGGATCTTCAGGCTTGTCCGACGTCACAAAAAGATCCG
TGGATGATTTTATCTCTACAGAAACACCTATTGCACTTAACAATCTCCTG
TGTAATGTTGGACCAGATGGTTGTAGAGCATTCGGCACAAGTGCAGGCGC
TGTTATTGCTTCTCCATCTACAATTGATCCAGACTATTACTACATGTGGA
CAAGAGACTCCGCCCTTGTGTTCAAAAACTTGATTGATCGTTTTACAGAA
ACTTACGATGCTGGATTACAAAGACGAATTGAACAATATATCACTGCTCA
AGTAACTTTACAAGGATTGAGTAATCCAAGTGGAAGTTTGGCAGATGGCT
CAGGACTAGGAGAGCCAAAGTTTGAACTAACCCTTAAGCCATTCACTGGG
AACTGGGGTAGACCACAAAGAGATGGTCCTGCTTTGAGAGCAATAGCCTT
AATCGGCTACTCAAAATGGTTAATCAACAATAACTACCAATCAACAGTTT
CAAATGTTATCTGGCCAATTGTTAGGAATGATTTGAACTACGTGGCTCAA
TACTGGAACCAGACCGGTTTCGACCTTTGGGAAGAGGTTAATGGCTCTTC
CTTTTTCACAGTGGCAAATCAGCATAGAGCTTTGGTTGAAGGAGCTACTT
TAGCGGCCACTCTCGGTCAGTCAGGTTCAGCTTACTCTTCTGTAGCTCCT
CAAGTACTTTGTTTTCTACAGAGATTCTGGGTATCTTCTGGTGGTTACGT
TGATTCTAACATTAACACAAATGAAGGGCGTACTGGCAAAGATGTGAATA
GCGTCCTTACCAGCATCCATACATTCGATCCTAATTTGGGTTGTGATGCC
GGGACGTTTCAACCTTGTTCTGACAAGGCTTTGAGCAATCTGAAAGTGGT
TGTTGATAGTTTCAGAAGCATCTACGGTGTAAACAAGGGTATTCCAGCTG
GTGCTGCCGTGGCTATCGGCAGATATGCAGAAGATGTCTACTATAATGGA
AATCCATGGTACTTGGCTACTTTTGCCGCAGCAGAACAGTTGTACGACGC
CATCTACGTTTGGAAAAAGACTGGTAGCATTACTGTTACAGCTACATCCT
TAGCATTTTTCCAAGAGTTAGTCCCAGGGGTCACAGCAGGCACGTACTCC
TCTTCTAGTTCAACCTTTACCAACATCATAAACGCTGTCTCCACCTATGC
CGACGGTTTTCTATCCGAGGCTGCCAAATACGTTCCTGCAGATGGTTCTC
TAGCTGAACAATTTGACAGAAATTCAGGTACTCCTCTGTCAGCAGTACAC
CTCACATGGAGTTACGCATCTTTTCTGACAGCAGCCGCGAGAAGAGCCGG
CATAGTTCCACCAAGTTGGGCCAATTCATCAGCCTCTACAATACCATCTA
CATGCTCAGGCGCTTCTGTTGTAGGGAGTTACTCTAGGCCAACCGCTACT
TCATTCCCACCTTCCCAAACTCCAAAACCAGGCGTACCTTCCGGAACACC
TTATACCCCACTCCCTTGCGCTACACCAACTTCAGTCGCAGTGACGTTTC
ACGAATTAGTTTCCACACAATTTGGTCACACAGTGAAAGTTGCAGGAAAT
GCCGCTGCTTTGGGCAATTGGTCAACTTCCGCAGCGGTAGCTTTGGACGC
TGTTAACTACAGAGATAATCATCCATTGTGGATTGGTACGGTCAACCTAG
AAGCTGGTGACGTCGTTGAGTATAAGTATATCATAGTTGGTCAAGATGGT
TCCGTCACTTGGGAGTCAGATCCTAATCATACTTACACTGTTCCTGCCGT
AGCTTGCGTCACACAAGTTGTGAAGGAAGATACTTGGCAATCTTAA

The expression cassette was linked with native S. cerevisiae URA3 gene. About 100 by of DNA derived from S. cerevisiae delta-sequence was placed on each of the flanks of the synthetic construct. The purpose of the delta-sequence is to target the integration events at the native delta sequences that are scattered around yeast chromosomes in many copies. For transformation, the construct, containing the elements outlined above was prepared free of bacterial vector sequences and used to transform an ura3 mutant derivative of industrial yeast strain FerMax Gold. A particular strain was selected from among such transformants based on its good performance under stress conditions.

The artificial sequence of synthetic Trichoderma reesei glucoamylase gene can be used to discriminate this strain from any other yeast strains. Another unique nucleotide sequence in the yeast is SEQ ID NO: 2, a 63 nucleotide remnant of Zygosaccharomyces rouxii acetamidase gene which is an artifact of vector construction path.

SEQ ID NO: 2
AGCTTTGTTTTTCGTGAATCTCTACGTCAGCTACTGTTTATCCGATGGTA
CTGTATCGCAACG

Additional strain engineering techniques can be employed according to the present teachings as will be appreciated by one of skill in the art. See for example Delft et al., US20110275130, and Berlin et al., US20120295319.

### Example 2

### Large Scale Fermentation to Make Yeast

In general the process uses a two stage seed train to build up cell mass for inoculation into the production tank. The first stage uses two to five Liters of any of several wake up media. It can be inoculated with a frozen starter culture. It is typically grown out to a dry cell weight of 5-15 g/L before being transferred to the second stage seed tank. The second seed stage uses a version of the production medium but with Glucose batched instead of metered into the tank. The concentration of Glucose used is within a range to provide the highest possible dry cell weight while also not producing ethanol at a concentration high enough to inhibit yeast growth. This range is 40-100 g/L. When cell growth in the second stage is completed as determined by either dry cell weight or the rate of cell respiration, the accumulated cell mass is inoculated into the main production tank. The second stage seed volume is typically around 10% of the starting production tank volume.

At inoculation of the production tank, a Glucose solution is fed to the culture at a rate that increases exponentially with time. The actual feed rate used is determined by the growth rate of the yeast strain being grown and the oxygen transfer capacity of the fermentation vessel. The feed continues through the growth phase. Temperature is controlled at a constant value within a range of 30-34° C. The pH of the fermentation is controlled with ammonia at a constant value within a range of 4.5 to 6.5. Agitation and tank pressure is enough to maintain positive dissolved oxygen.

At the end of growth phase in the production tank, a wind down period of typically three to five hours is used to transition the culture out of rapid growth and prepare it for cell recovery. The wind down consists of a rapid reduction in Glucose feed rate to put the yeast culture under carbon limitation. The primary purpose of the wind down is to allow the completion of the last budding cycle and the production of reserve carbohydrates that are stored in the cells. The most important of these carbohydrates is thought to be Trehalose. To survive the recovery and drying process to ADY product, it is desirable that the yeast cells have stored enough Trehalose to act as thermo protector and carbohydrate source. At the end of fermentation Trehalose can comprise 15-20% of dry cell weight. Yeast cells make Trehalose under carbon limiting conditions. This limit is desirably severe enough to stop budding, but not so restrictive as to prevent forming storage products. A typical yeast production fermentation with wind down is 24 to 26 hours in length.

Both the second stage seed and production tanks can use an inorganic defined medium such as that listed below. The formulation of the medium was designed around the composition of yeast cells and set to a strength so as to provide enough nutrients to produce a dry cell weight of around 100 g/Kg. The medium can use food grade, Kosher, and Halal approved raw materials.

The tank medium includes: Potassium phosphate-monobasic, Ammonium phosphate-dibasic, Ammonium sulfate, Magnesium sulfate-heptahydrate, Ferrous sulfate-heptahtdrate, Calcium hydroxide, Glucose, MnSO4, CuSO4*5H2O, ZnSO4*7H2O, Na2MoO4*2H2O, D-Pantothenic Acid, Hemicalcium Salt, Thiamine-HCl, Riboflavin, Nicotinic Acid, Pyridoxine-HCl, D-Botin, and Folic Acid.

### Example 3

### Recovery

At the end of fermentation, the broth is cooled (generally less than 15° C., typically 8-15° C.) as quickly as possible. pH control at the fermentation setpoint remains on (5.0). After cooling down, the cooled broth may either be fed directly to the centrifuge, or first to a drop tank. Ideally, the harvest broth should be processed on the centrifuge immediately after cooling down is complete.

Centrifugation may begin before the cool-down target temperature is reached. Centrifugation serves to remove spent media, wash, and concentrate the yeast cells, producing the cream—a concentrated yeast slurry that can be pumped. For the cream process, a minimum of 1 centrifuge pass is usually employed in order to achieve the concentration factor that is desired. However, washing the cream is not needed to process the cream through to ADY. This particular strain can achieve a cream DCW (Dry Cell Weight) of up to 230 g/kg (measured by drying in a microwave) or about 75-80% PCV (Packed Cell Volume, spun at 10,000 g*min)—beyond this, the cream may not be pump transferable. A range of 190-230 g/kg is typical for the cream, but the final percentage can be maximized to efficiently remove spent media/wash the yeast and reduce the shipping cost and filtration cycle times.

Interestingly, there is no evidence that the viability and long-term stability of the ADY product is affected by the number of washes—although two washes is generally employed to achieve complete decolorizing of the cream and to reduce the total dry solids (DS %, measured as weight/weight %) of the centrate to minimal levels. There is typically a 5-fold reduction in the DS % of the fermentation supernatant or wash water after each pass though the centrifuge, indicating that by the 3^{rd} pass soluble or suspended components (other than the viable yeast, of course) from the fermentation broth are essentially removed: centrate pass 1=5%; centrate pass 2 (Wash 1)=1%; centrate pass 3 (Wash 2)=0.2%). Washes should be done with cold (15° C. or less) process water which is either added after all cream has been collected, or added to the cream destination tank beforehand. The washes are achieved by re-suspending the cream to about the original DCW of the harvest broth and again passing thru the centrifuge. The final cream is transferred to a hold tank and stored under cooling and agitation for up to 2 weeks or more before further processing.

The initial harvest broth pH is approximately 5.0. The pH of the cream is not maintained or adjusted during wash and concentration steps. The cream pH tends to increase 0.1-0.2 units after each pass to a final value of 5.5-5.4. During storage and holding period, the pH tends to drifts down to 4.2-4.6. The cream pH is not maintained during storage, because it is typically steady after reaching 4.2-4.6 range. Below pH 4.0 is thought to be harmful to yeast viability, although it is not yet known what excursion outside this range for a short period of time will have on long term stability of the ADY product. Although it should ideally be held under constant cooling and agitation, cream stored cold (4-10° C.) in totes during short (1-2 week) periods, with brief agitation beforehand to resuspend settled yeast, can be processed to ADY product.

### Example 4

### Fluid Bed Formulation

The yeast cream with dry solids of 190-230 g/kg is dewatered on a membrane filter press (or rotary drum filter) to produce a wet cake. A membrane type filter press is needed so the moisture content can be controlled consistently by squeezing. The media used for filtration is Polypropylene cloth with empirically chosen pore size. No formulation ingredient or admix is required for cream filtration. The filtration pressure is controlled for optimum throughput. Cake squeeze (air or water as media) is followed after filtration to further dewater the cake inside chambers. The wet cake dry solid is between 350-390 g/kg. The filtration is done at cold temperature. Wet cakes are broken using an auger and immediately transported to an extruder.

A potential alternative processing option is to start with fermentation harvest broth at a solids level of 90-100 g/Kg and then dewater using a membrane filter press (or rotary drum filter) to the same conditions stated above.

The wet cake harvested from the filter press should be processed immediately to avoid viability loss. The wet cake needs to be broken to manageable size pieces before being fed directly to a low pressure screw extruder. The function of the extruder is to form wet cake into noodles, with points of breakage or “notches” so that they break into cylindrical particles. This is accomplished by using counter-rotating twin screws to force the wet cake though a radial or dome shaped plate with die holes that are of the appropriate diameter (e.g. 800 μm). The noodles are collected or transported in the product bowl of a fluidized bed dryer and immediately sent to a dryer. There is little or no loss of viability during the extrusion process.

The broken noodles are dried using a fluid bed dryer. Drying is conducted in two phases. After all noodles are loaded into the dryer the first phase of drying is done to drive off the free extracellular moisture between yeast cells, and where the yeasts are preserved by evaporative cooling. Once the extracellular moisture is driven off, the second phase of drying begins, where the moisture from inside the cells is removed. During this phase the inlet air temperature is reduced to avoid overheating the product. The dryer cycle is completed at target product bed temperature and relative humidity. Air flow throughout the process is set to maintain fluidization of the noodles. ADY (5-8% moisture) is unloaded from dryer and immediately packaged.

### Example 5

### Spray Drying Formulation

As an alternative to fluid bed drying and formulation, the yeast cream with dry solids of 90-230 g/Kg DCW can be spray dried. The cream may or may not be washed/diafiltered with water. The cream can be safely refrigerated (less than 15 C) until dried. If any settling occurs during, agitation can be used to disperse the solids. The cream is prepared for drying using an empirically chosen recipe that may include the addition of different binding and/or agglomerating agents and/or drying aids (i.e. Maltrin).

The prepared cream can be pumped up to the top of the tower dryer where various nozzle configurations and pressures (between 500-3000 psig) can be used. Different inlet air temperatures from 140 F-190 F can be used to generate moisture levels from 5-25%. Varying the nozzle and pressure will also influence the final product moisture and particle size. The dried powder is collected from/at the bottom of the tower and directed to a fluid bed drier to complete the drying and remove fine particles. The fine particles can be recycled back into the top of the tower to facilitate growth of larger particles. The dried product is collected and packaged.

### Example 6

### Large Scale Fermentation to Make Ethanol

Successful use of the genetically engineered yeast in active dry form was achieved by performing an 807,000 gallon commercial dry grind ethanol fermentation, and comparing the ethanol produced to a conventional yeast fermentation containing a full conventional dose of glucoamylase. The demonstration began with propagation. 40 kilograms of active dry yeast made by fluid bed drying was added to a 20,000 gallon yeast propagation tank that was prepared with a conventional mixture of ground corn liquefact, water, urea, protease, glucoamylase, zinc sulfate, and antibiotics. This mixture was controlled at a temperature of 31-32 C and allowed to ferment for 6-8 hrs. Cell counts, viability, and ethanol production were similar between the two yeasts during this propagation process. At the completion of the 6-8 hr propagation time, the entire contents of the propagation tank were sent to the main fermentor.

The main 807,000 gallon fermentor was prepared in the typical dry grind process using ground corn liquefact, urea, protease, antibiotics, and glucoamylase. For the genetically engineered yeast of the present teachings, the amount of exogenous glucoamylase was only 27% of the amount needed for the conventional yeast and its full dose of glucoamylase. This mixture is allowed to ferment for 50-60 hrs.

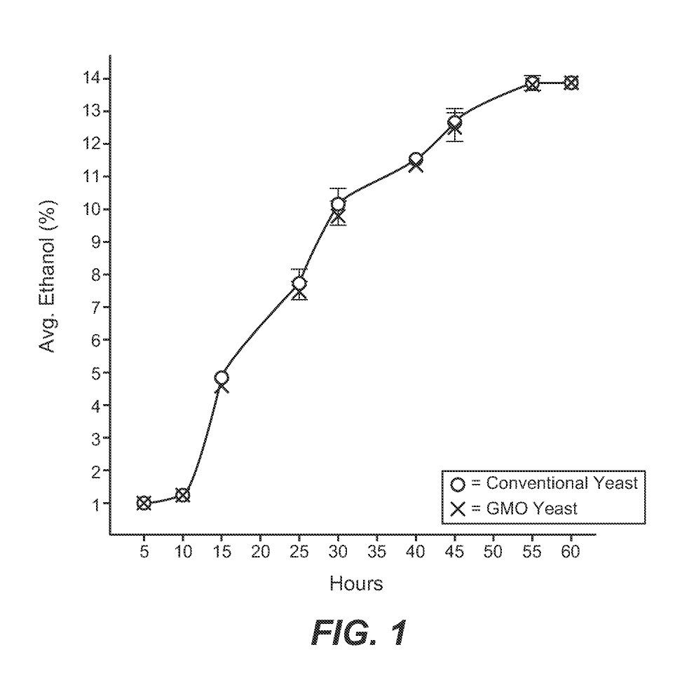

The results of this experiment are shown in FIG. 1. Here, the ethanol produced indicates that the genetically modified yeast is able to hydrolyze all the starch to glucose, but with only 27% of the normal dose of glucoamylase. The genetically modified yeast was thus able to ferment glucose to the same amount of ethanol in the defined process time as the conventional yeast.

SEQUENCE LISTING

```
SEQ ID NO: 1
ATGCTACTCCAAGCATTCCTTTTTCTGTTAGCAGGATTTGCTGCCAAAATCTCTGCTAGACCTG
GATCTTCAGGCTTGTCCGACGTCACAAAAAGATCCGTGGATGATTTTATCTCTACAGAAACACC
TATTGCACTTAACAATCTCCTGTGTAATGTTGGACCAGATGGTTGTAGAGCATTCGGCACAAGT
GCAGGCGCTGTTATTGCTTCTCCATCTACAATTGATCCAGACTATTACTACATGTGGACAAGAG
ACTCCGCCCTTGTGTTCAAAAACTTGATTGATCGTTTTACAGAAACTTACGATGCTGGATTACA
AAGACGAATTGAACAATATATCACTGCTCAAGTAACTTTACAAGGATTGAGTAATCCAAGTGGA
AGTTTGGCAGATGGCTCAGGACTAGGAGAGCCAAAGTTTGAACTAACCCTTAAGCCATTCACTG
GGAACTGGGGTAGACCACAAAGAGATGGTCCTGCTTTGAGAGCAATAGCCTTAATCGGCTACTC
AAAATGGTTAATCAACAATAACTACCAATCAACAGTTTCAAATGTTATCTGGCCAATTGTTAGG
AATGATTTGAACTACGTGGCTCAATACTGGAACCAGACCGGTTTCGACCTTTGGGAAGAGGTTA
ATGGCTCTTCCTTTTTCACAGTGGCAAATCAGCATAGAGCTTTGGTTGAAGGAGCTACTTTAGC
GGCCACTCTCGGTCAGTCAGGTTCAGCTTACTCTTCTGTAGCTCCTCAAGTACTTTGTTTTCTA
CAGAGATTCTGGGTATCTTCTGGTGGTTACGTTGATTCTAACATTAACACAAATGAAGGGCGTA
CTGGCAAAGATGTGAATAGCGTCCTTACCAGCATCCATACATTCGATCCTAATTTGGGTTGTGA
TGCCGGGACGTTTCAACCTTGTTCTGACAAGGCTTTGAGCAATCTGAAAGTGGTTGTTGATAGT
TTCAGAAGCATCTACGGTGTAAACAAGGGTATTCCAGCTGGTGCTGCCGTGGCTATCGGCAGAT
ATGCAGAAGATGTCTACTATAATGGAAATCCATGGTACTTGGCTACTTTTGCCGCAGCAGAACA
GTTGTACGACGCCATCTACGTTTGGAAAAAGACTGGTAGCATTACTGTTACAGCTACATCCTTA
GCATTTTTCCAAGAGTTAGTCCCAGGGGTCACAGCAGGCACGTACTCCTCTTCTAGTTCAACCT
TTACCAACATCATAAACGCTGTCTCCACCTATGCCGACGGTTTTCTATCCGAGGCTGCCAAATA
CGTTCCTGCAGATGGTTCTCTAGCTGAACAATTTGACAGAAATTCAGGTACTCCTCTGTCAGCA
GTACACCTCACATGGAGTTACGCATCTTTTCTGACAGCAGCCGCGAGAAGAGCCGGCATAGTTC
CACCAAGTTGGGCCAATTCATCAGCCTCTACAATACCATCTACATGCTCAGGCGCTTCTGTTGT
AGGGAGTTACTCTAGGCCAACCGCTACTTCATTCCCACCTTCCCAAACTCCAAAACCAGGCGTA
CCTTCCGGAACACCTTATACCCCACTCCCTTGCGCTACACCAACTTCAGTCGCAGTGACGTTTC
ACGAATTAGTTTCCACACAATTTGGTCACACAGTGAAAGTTGCAGGAAATGCCGCTGCTTTGGG
CAATTGGTCAACTTCCGCAGCGGTAGCTTTGGACGCTGTTAACTACAGAGATAATCATCCATTG
TGGATTGGTACGGTCAACCTAGAAGCTGGTGACGTCGTTGAGTATAAGTATATCATAGTTGGTC
AAGATGGTTCCGTCACTTGGGAGTCAGATCCTAATCATACTTACACTGTTCCTGCCGTAGCTTG
CGTCACACAAGTTGTGAAGGAAGATACTTGGCAATCTTAA
SEQ ID NO: 2
AGCTTTGTTTTTCGTGAATCTCTACGTCAGCTACTGTTTATCCGATGGTACTGTATCGCAACG
Nocardiopsis sp. (NRRL 18262, Strain 10R)
SEQ ID NO: 3
      1 mrpspvasai gtgalafgla laaapgalaa sgplpqaptp eaeavsmkea lqrdldltps
    61 eaeslltaqd tafeideaaa eaagdayggs vfdtetldlt vlvtdaaave aveaagaeae
  121 vvdfgiegld eivedlndag tvpgvvgwyp dvegdtvvle vlegsgadvd gllaeagvda
  181 savevattde qpqvyadiig glaytmggrc svgfaatnsa gqpgfvtagh cgtvgtqvsi
  241 gngrgvfers vfpgndaafv rgtsnftltn lvsrynsggy atvsgssaap igssvcrsgs
  301 ttgwhcgtiq argqsvsypq gtvtnmtrts vcaepgdsgg sfisgtqaqg vtsggsgncr
  361 tggttyyqev npminswgvr lrt
Citrobacter braakii phytase
SEQ ID NO: 4
      1: EEQNG MKLER VVIVS RHGVR APTKF TPIMK NVTPD QWPQW DVPLG WLTPR
    51: GGELV SELGQ YQRLW FTSKG LLNNQ TCPSP GQVAV IADTD QRTRK TGEAF
  101: LAGLA PKCQI QVHYQ KDEEK NDPLF NPVKM GKCSF NTLQV KNAIL ERAGG
  151: NIELY TQRYQ SSFRT LENVL NFSQS ETCKT TEKST KCTLP EALPS ELKVT
  201: PDNVS LPGAW SLSST LTEIF LLQEA QGMPQ VAWGR ITGEK EWRDL LSLHN
  251: AQFDL LQRTP EVARS RATPL LDMID TALLT NGTTE NRYGI KLPVS LLFIA
  301: GHDTN LANLS GALDL NWSLP GQPDN TPPGG ELVFE KWKRT SDNTD WVQVS
  351: FVYQT LRDMR DIQPL SLEKP AGKVD LKLIA CEEKN SQGMC SLKSF SRLIK
  401: EIRVP ECAVT E
Aspergillus niger phytase (DSM)
SEQ ID NO: 5
      1: ASRNQ SSCDT VDQGY QCFSE TSHLW GQYAP FFSLA NESVI SPEVP AGCRV
    51: TFAQV LSRHG ARYPT DSKGK KYSAL IEEIQ QNATT FDGKY AFLKT YNYSL
  101: GADDL TPFGE QELVN SGIKF YQRYE SLTRN IVPFI RSSGS SRVIA SGKKF
  151: IEGFQ STKLK DPRAQ PGQSS PKIDV VISEA SSSNN TLDPG TCTVF EDSEL
  201: ADTVE ANFTA TFVPS IRQRL ENDLS GVTLT DTEVT YLMDM CSFDT ISTST
  251: VDTKL SPFCD LFTHD EWINY DYLQS LKKYY GHGAG NPLGP TQGVG YANEL
  301: IARLT HSPVH DDTSS NHTLD SSPAT FPLNS TLYAD FSHDN GIISI LFALG
  351: LYNGT KPLST TTVEN ITQTD GFSSA WTVPF ASRLY VEMMQ CQAEQ EPLVR
  401: VLVND RVVPL HGCPV DALGR CTRDS FVRGL SFARS GGDWA ECFA
Rhizomucor pusillus alpha-amylase
SEQ ID NO: 6
      1: SPLPQ QQRYG KRATS DDWKS KAIYQ LLTDR FGRAD DSTSN CSNLS NYCGG
    51: TYEGI TKHLD YISGM GFDAI WISPI PKNSD GGYHG YWATD FYQLN SNFGD
  101: ESQLK ALIQA AHERD MYVML DVVAN HAGPT SNGYS GYTFG DASLY HPKCT
  151: IDYND QTSIE QCWVA DELPD IDTEN SDNVA ILNDI VSGWV GNYSF DGIRI
  201: DTVKH IRKDF WTGYA EAAGV FATGE VFNGD PAYVG PYQKY LPSLI NYPMY
  251: YALND VFVSK SKGFS RISEM LGSNR NAFED TSVLT TFVDN HDNPR FLNSQ
  301: SDKAL FKNAL TYVLL GEGIP IVYYG SEQGF SGGAD PANRE VLWTT NYDTS
  351: SDLYQ FIKTV NSVRM KSNKA VYMDI YVGDN AYAFK HGDAL VVLNN YGSGS
  401: TNQVS FSVSG KFDSG ASLMD IVSNI TTTVS SDGTV TFNLK DGLPA IFTSA
Taleromyces emersonii gluco-amylase
SEQ ID NO: 7
      1: ATGSL DSFLA TETPI ALQGV LNNIG PNGAD VAGAS AGIVV ASPSR SDPNY
    51: FYSWT RDAAL TAKYL VDAFN RGNKD LEQTI QQYIS AQAKV QTISN PSGDL
  101: STGGL GEPKF NVNET AFTGP WGRPQ RDGPA LRATA LIAYA NYLID NGEAS
  151: TADEI IWPIV QNDLS YITQY WNSST FDLWE EVEGS SFFTT AVQHR ALVEG
  201: NALAT RLNHT CSNCV SQAPQ VLCFL QSYWT GSYVL ANFGG SGRSG KDVNS
  251: ILGSI HTFDP AGGCD DSTFQ PCSAR ALANH KVVTD SFRSI YAINS GIAEG
  301: SAVAV GRYPE DVYQG GNPWY LATAA AAEQL YDAIY QWKKI GSISI TDVSL
  351: PFFQD IYPSA AVGTY NSGST TFNDI ISAVQ TYGDG YLSIV EKYTP SDGSL
  401: TEQFS RTDGT PLSAS ALTWS YASLL TASAR RQSVV PASWG ESSAS SVLAV
  451: CSATS ATGPY STATN TVWPS SGSGS STTTS SAPCT TPTSV AVTFD EIVST
  501: SYGET IYLAG SIPEL GNWST ASAIP LRADA YTNSN PLWYV TVNLP PGTSF
  551: EYKFF KNQTD GTIVW EDDPN RSYTV PAYCG QTTAI LDDSW Q
Trametes cingulata gluco-amylase
SEQ ID NO: 8
      1: QSSAA DAYVA SESPI AKAGV LANIG PSGSK SNGAK ASDTP ASXIA SPSTS
    51: NPNYL YTWTR DSSLV FKALI DQFTT GEDTS LRTLI DEFTS AEAIL QQVPN
  101: PSGTV STGGL GEPKF NIDET AFTDA WGRPQ RDGPA LRATA IITYA NWLLD
  151: NKNTT YVTNT LWPII KLDLD YVASN WNQST FDLWE EINSS SFFTT AVQHR
  201: ALREG ATFAN RIGQT SVVSG YTTQA NNLLC FLQAS YWNPT GGYIT ANTGG
  251: GRSGK DANTV LTSIH TFDPA AGCDA VTFQP CSDKA LSNLK VYVDA FRSIY
  301: SINSG IASNA AVATG RYPED SYMGG NPWYL TTSAV AEQLY DALIV WNKLG
  351: ALNVT STSLP FFQQF SSGVT VGTYA SSSST FKTLT SAIKT FADGF LAVNA
  401: KYTPS NGGLA EQYSR SNGSP VSAVD LTWSY AAALT SFAAR SGKTY ASWGA
  451: AGLTV PTTCS GSGGA GTVAV TFNVQ ATTVF GENIY ITGSV PALQN WSPDN
  501: ALILS AANYP TWSIT VNLPA STTIE YKYIR KFNGA VTWES DPNNS ITTPA
  551: SGTFT QNDTW R
Aspergillus kawachii alpha-amylase
SEQ ID NO: 9
      1: LSAAE WRTQS IYFLL TDRFG RTDNS TTATC NTGDQ IYCGG SWQGI INHLD
    51: YIQGM GFTAI WISPI TEQLP QDTSD GEAYH GYWQQ KIYNV NSNFG TADDL
  101: KSLSD ALHAR GMYLM VDVVP NHMGY AGNGN DVDYS VFDPF DSSSY FHPYC
  151: LITDW DNLTM VQDCW EGDTI VSLPD LNTTE TAVRT IWYDW VADLV SNYSV
  201: DGLRI DSVEE VEPDF FPGYQ EAAGV YCVGE VDNGN PALDC PYQKY LDGVL
  251: NYPIY WQLLY AFESS SGSIS NLYNM IKSVA SDCSD PTLLG NFIEN HDNPR
  301: FASYT SDYSQ AKNVL SYIFL SDGIP IVYAG EEQHY SGGDV PYNRE ATWLS
  351: GYDTS AELYT WIATT NAIRK LAISA DSDYI TYAND PIYTD SNTIA MRKGT
  401: SGSQI ITVLS NKGSS GSSYT LTLSG SGYTS GTKLI EAYTC TSVTV DSNGD
  451: IPVPM ASGLP RVLLP ASVVD SSSLC GGSGN TTTTT TAATS TSKAT TSSSS
  501: SSAAA TTSSS CTATS TTLPI TFEEL VTTTY GEEVY LSGSI SQLGE WDTSD
  551: AVKLS ADDYT SSNPE WSVTV SLPVG TTFEY KFIKV DEGGS VTWES DPNRE
  601: YTVPE CGSGS GETVV DTWR
Humicola grisea gluco-amylase
SEQ ID NO: 10
MHTFSKLLVLGSAVQSALGRPHGSSRLQER
AAVDTFINTEKPIAWNKLLANIGPNGKAAPGAAAGVVIASPSRTDPPYFF
TWTRDAALVLTGIIESLGHNYNTTLQTVIQNYVASQAKLQQVSNPSGTFADGSGLGEAKFNVDLTAFTGE
WGRPQRDGPP
LRAIALIQYAKWLIANGYKSTAKSVVWPVVKNDLAYTAQYWNETGFDLWEEVPGSSFFTIASSHRALTEG
AYLAAQLDTE
CRACTTVAPQVLCFQQAFWNSKGNYVVSNINGGEYRSGKDANSILASIHNFDPEAGCDNLTFQPCSERA
LANHKAYVDSF
RNLYAINKGIAQGKAVAVGRYSEDVYYNGNPWYLANFAAAEQLYDAIYVWNKQGSITVTSVSLPFFRDLV
SSVSTGTYSK
SSSTFTNIVNAVKAYADGFIEVAAKYTPSNGALAEQYDRNTGKPDSAADLTWSYSAFLSAIDRRAGLVPP
SWRASVAKSQ
LPSTCSRIEVAGTYVAATSTSFPSKQTPNPSAAPSPSPYPTACADASEVYVTFNERVSTAWGETIKVVGN
VPALGNWDTS
KAVTLSASGYKSNDPLWSITVPIKATGSAVQYKYIKVGTNGKITWESDPNRSITLQTASSAGKCAAQTVND
SWR
Saccharomycopsis fibuligera gluco-amylase AE8
SEQ ID NO: 11
      1: AYPSF EAYSN YKVDR TDLET FLDKQ KDVSL YYLLQ NIAYP EGQFN DGVPG
    51: TVIAS PSTSN PDYYY QWTRD SAITF LTVLS ELEDN NFNTT LAKAV EYYIN
  101: TSYNL QRTSN PSGSF DDENH KGLGE PKFNT DGSAY TGAWG RPQND GPALR
  151: AYAIS RYLND VNSLN KGKLV LTDSG DINFS STEDI YKNII KPDLE YVIGY
  201: WDSTG FDLWE ENQGR HFFTS LVQQK ALAYA VDIAK SFDDG DFANT LSSTA
  251: STLES YLSGS DGGFV NTDVN HIVEN PDLLQ QNSRQ GLDSA TYIGP LLTHD
  301: IGESS STPFD VDNEY VLQSY YLLLE DNKDR YSVNS AYSAG AAIGR YPEDV
  351: YNGDG SSEGN PWFLA TAYAA QVPYK LVYDA KSASN DITIN KINYD FFNKY
  401: IVDLS TINSG YQSSD SVTIK SGSDE FNTVA DNLVT FGDSF LQVIL DHIND
  451: DGSLN EQLNR NTGYS TSAYS LTWSS GALLE AIRLR NKVKA LA
Aspergillus niger alpha-amylase
SEQ ID NO: 12
      1: LSAAE WRTQS IYFLL TDRFG RTDNS TTATC DTGDQ IYCGG SWQGI INHLD
    51: YIQGM GFTAI WISPI TEQLP QDTAD GEAYH GYWQQ KIYDV NSNFG TADDL
  101: KSLSD ALHAR GMYLM VDVVP NHMGY AGNGN DVDYS VFDPF DSSSY FHPYC
  151: LITDW DNLTM VQDCW EGDTI VSLPD LNTTE TAVRT IWYDW VADLV SNYSV
  201: DGLRI DSVLE VEPDF FPGYQ EAAGV YCVGE VDNGN PALDC PYQKV LDGVL
  251: NYPIY WQLLY AFESS SGSIS NLYNM IKSVA SDCSD PTLLG NFIEN HDNPR
  301: FASYT SDYSQ AKNVL SYIFL SDGIP IVYAG EEQHY SGGKV PYNRE ATWLS
  351: GYDTS AELYT WIATT NAIRK LAISA DSAYI TYAND AFYTD SNTIA MRKGT
  401: SGSQV ITVLS NKGSS GSSYT LTLSG SGYTS GTKLI EAYTC TSVTV DSSGD
  451: IPVPM ASGLP RVLLP ASVVD SSSLC GGSGR LYVE
Trichoderma reesei trehalase
SEQ ID NO: 13
      1: TLVDR VTKCL SRHDG SDAES HFSKN VYKTD FAGVT WDEDN WLLST TQLKQ
    51: GAFEA RGSVA NGYLG INVAS VGPFF EVDTE EDGDV ISGWP LFSRR QSFAT
  101: VAGFW DAQPQ MNGTN FPWLS QYGSD TAISG IPHWS GLVLD LGGGT YLDAT
  151: VSNKT ISHFR STYDY KAGVL SWSYK WTPKG NKGSF DISYR LFANK LHVNQ
  201: AVVDM QVTAS KNVQA SIVNV LDGFA AVRTD FVESG EDGSA IFAAV RPNGV
  251: ANVTA YVYAD ITGSG GVNLS SRKIV HNKPY VHANA SSIAQ AVPVK FAAGR
  301: TVRVT KFVGA ASSDA FKNPK QVAKK AAAAG LSNGY TKSLK AHVEE WATVM
  351: PESSV DSFAD PKTGK LPADS HIVDS AIIAV TNTYY LLQNT VGKNG IKAVD
  401: GAPVN VDSIS VGGLT SDSYA GQIFW DADLW MQPGL VAAHP EAAER ITNYR
  451: LAYGQ AKENV KTAYA GSQNE TFFSA SAAVF PWTSG RYGNC TATGP CWDYE
  501: YHLNG DIGIS LVNQW VVNGD TKDFE KNLFP VYDSV AQLYG NLLRP NKTSW
  551: TLTNM TDPDE YANHV DAGGY TMPLI AETLQ KANSF RQQFG IEQNK TWNDM
  601: ASNVL VLREN GVTLE FTAMN GTAVV KQADV IMLTY PLSYG TNYSA QDALN
  651: DLDYY ANKQS PDGPA MTYAF FSIVA NEISP SGCSA YTYAQ NAFKP YVRAP
  701: FYQIS EQLID DASVN GGTHP AYPFL TGHGG AHQVV LFGYL GLRLV PDDVI
  751: HIEPN LPPQI PYLRY RTFYW RGWPI SAWSN YTHTT LSRAA GVAAL EGADQ
  801: RFARK PITIH AGPEQ DPTAY RLPVK GSVVI PNKQI GSQQT YAGNL VQCHA
  851: ASSPN DYVPG QFPIA AVDGA TSTKW QPASA DKVSS ITVSL DKEDV GSLVS
  901: GFHFD WAQAP PVNAT VIFHD EALAD PATAL ASAHK HNSKY TTVTS LTNIE
  951:LSDPY VSTKD LNAIA IPIGN TTNVT LSHPV AASRY ASLLI VGNQG LDPVD
1001: VKAKN GTGAT VAEWA IFGHG KEHSG KPSSH SKRRL NVRTA ATLSN PRSFM
1051: RRRL
Bacillus deramificans pullulanase
SEQ ID NO: 14
      1: DGNTT TIIVH YFRPA GDYQP WSLWM WPKDG GGAEY DFNQP ADSLG AVASA
    51: DIPGN PSQVG IIVRT QDWTK DVSAD RYIDL SKGNE VWLVE GNSQI FYSEK
  101: DAEDA AKPAV SNAYL DASNQ VLVKL SQPLT LGEGA SGFTV HDDTA NKDIP
  151: VTSVK DASLG QDVTA VLAGT FQHIF GGSDW APDNH STLLK KVTNN LYQFS
  201: GDLPE GNYQY KVALN DSWNN PSYPS DNINL TVPAG GAHVT FSYIP STHAV
  251: YDTIN NPNAD LQVES GVKTD LVTVT LGEDP DVSHT LSIQT DGYQA KQVIP
  301: RNVLN SSQYY YSGDD LGNTY TQKAT TFKVW APTST QVNVL LYDSA TGSVT
  351: KIVPM TASGH GVWEA TVNQN LENWY YMYEV TGQGS TRTAV DPYAT AIAPN
  401: GTRGM IVDLA KTDPA GWNSD KHITP KNIED EVIYE MDVRD FSIDP NSGMK
  451: NKGKY LALTE KGTKG PDNVK TGIDS LKQLG ITHVQ LMPVF ASNSV DETDP
  501: TQDNW GYDPR NYDVP EGQYA TNANG NARIK EFKEM VLSLH REHIG VNMDV
  551: VYNHT FATQI SDFDK IVPEY YYRTD DAGNY TNGSG TGNEI AAERP MVQKF
  601: IIDSL KYWVN EYHID GFRFD LMALL GKDTM SKAAS ELHAI NPGIA LYGEP
  651: WTGGT SALPD DQLLT KGAQK GMGVA VFNDN LRNAL DGNVF DSSAQ GFATG
  701: ATGLT DAIKN GVEGS INDFT SSPGE TINYV TSHDN YTLWD KIALS NPNDS
  751: EADRI KMDEL AQAVV MTSQG VPFMQ GGEEM LRTKG GNDNS YNAGD AVNEF
  801: DWSRK AQYPD VFNYY SGLIH LRLDH PAFRM TTANE INSHL QFLNS PENTV
  851: AYELT DHVNK DKWGN IIVVY NPNKT VATIN LPSGK WAINA TSGKV GESTL
  901: GQAEG SVQVP GISMM ILHQE VSPDH GKK
Buttiauxella sp. Phytase:
SEQ ID NO: 15
      1: NDTPA SGYQV EKVVI LSRHG VRAPT KMTQT MRDVT PNTWP EWPVK LGYIT
    51: PRGEH LISLM GGFYR QKFQQ QGILS QGSCP TPNSI YVWAD VDQRT LKTGE
  101: AFLAG LAPQC GLTIH HQQNL EKADP LFHPV KAGTC SMDKT QVQQA VEKEA
  151:QTPID NLNQH YIPFL ALMNT TLNFS TSAWC QKHSA DKSCD LGLSM PSKLS
  201: IKDNG NKVAL DGAIG LSSTL AEIFL LEYAQ GMPQA AWGNI HSEQE WASLL
  251: KLHNV QFDLM ARTPY IARHN GTPLL QAISN ALNPN ATESK LPDIS PDNKI
  301: LFIAG HDTNI ANIAG MLNMR WTLPG QPDNT PPGGA LVFER LADKS GKQYV
  351: SVSMV YQTLE QLRSQ TPLSL NQPAG SVQLK IPGCN DQTAE GYCPL STFTR
  401: VVSQS VEPGC QLQ
Trichoderma reesei protease
SEQ ID NO: 16
      1: LPTEG QKTAS VEVQY NKNYV PHGPT ALFKA KRKYG APISD NLKSL VAARQ
    51: AKQAL AKRQT GSAPN HPSDS ADSEY ITSVS IGTPA QVLPL DFDTG SSDLW
  101: VFSSE TPKSS ATGHA IYTPS KSSTS KKVSG ASWSI SYGDG SSSSG DVYTD
  151: KVTIG GFSVN TQGVE SATRV STEFV QDTVI SGLVG LAFDS GNQVR PHPQK
  201: TWFSN AASSL AEPLF TADLR HGQNG SYNFG YIDTS VAKGP VAYTP VDNSQ
  251: GFWEF TASGY SVGGG KLNRN SIDGI ADTGT TLLLL DDNVV DAYYA NVQSA
  301: QYDNQ QEGVV FDCDE DLPSF SFGVG SSTIT IPGDL LNLTP LEEGS STCFG
  351: GLQSS SGIGI NIFGD VALKA ALVVF DLGNE RLGWA QK
full-length Aspergillus clavatus alpha-amylase
SEQ ID NO: 17
MKLLALTTAFALLGKGVFGLTPAEWRGQSIYFLITDRFARTDGSTTAPCDLSQRAYCGGSWQGIIKQLDYI
QGMGFTAIWITPITEQIPQDTAEGSAFHGYWQKDIYNVNSHFGTADDIRALSKALHDRGMYLMIDVVANH
MGYNGPGASTDFSTFTPFNSASYFHSYCPINNYNDQSQVENCWLGDNTVALADLYTQHSDVRNIWYSW
IKEIVGNYSADGLRIDTVKHVEKDFWTGYTQAAGVYTVGEVLDGDPAYTCPYQGYVDGVLNYPIYYPLLR
AFESSSGSMGDLYNMINSVASDCKDPTVLGSFIENHDNPRFASYTKDMSQAKAVISYVILSDGIPIlYSGQ
EQHYSGGNDPYNREAIWLSGYSTTSELYKFIATTNKIRQLAISKDSSYLTSRNNPFYTDSNTIAMRKGSGG
SQVITVLSNSGSNGGSYTLNLGNSGYSSGANLVEVYTCSSVTVGSDGKIPVPMASGLPRVLVPASWMS
GSGLCGSSSTTTLVTATTTPTGSSSSTTLATAVTTPTGSCKTATTVPVVLEESVRTSYGENIFISGSIPQLG
SWNPDKAVALSSSQYTSSNPLWAVTLDLPVGTSFEYKFLKKEQNGGVAWENDPNRSYTVPEACAGTSQ
KVDSSWR*
Full length Trichoderma reesi engineered glucoamylase exemplified in Example 1
SEQ ID NO: 18
MHVLSTAVLLGSVAVQKVLGRPGSSGLSDVTKRSVDDFISTETPIALNNLLCNVGPDGCRAFGTSAGAVI
ASPSTIDPDYYYMWTRDSALVFKNLIDRFTETYDAGLQRRIEQYITAQVTLQGLSNPSGSLADGSGLGEP
KFELTLKPFTGNWGRPQRDGPALRAIALIGYSKWLINNNYQSTVSNVIWPIVRNDLNYVAQYWNQTGFDL
WEEVNGSSFFTVANQHRALVEGATLAATLGQSGSAYSSVAPQVLCFLQRFWVSSGGYVDSNINTNEGRTG
KDVNSVLTSIHTFDPNLGCDAGTFQPCSDKALSNLKVVVDSFRSIYGVNKGIPAGAAVAIGRYAEDVYYN
GNPWYLATFAAAEQLYDAIYVWKKTGSITVTATSLAFFQELVPGVTAGTYSSSSSTFTNIINAVSTYADG
FLSEAAKYVPADGSLAEQFDRNSGTPLSAVHLTWSYASFLTAAARRAGIVPPSWANSSASTIPSTCSGAS
VVGSYSRPTATSFPPSQTPKPGVPSGTPYTPLPCATPTSVAVTFHELVSTQFGHTVKVAGNAAALGNWST
SAAVALDAVNYRDNHPLWIGTVNLEAGDVVEYKYIIVGQDGSVTWESDPNHTYTVPAVACVTQVVKEDTW
QS
Rhizopus oryzae alpha-amylase
SEQ ID NO: 19
MKSFLSLLCSVFLLPLVVQS
VPVIKRASASDWENRVIYQLLTDRFAKSTDDTNGCNNLSDYCGGTFQGIINHLDYIAGMGFDAIWISPIP
KNANGGYHGYWATDFSQINEHFGTADDLKKLVAAAHAKNMYVMLDVVANHAGIPSSGGDYSGYTFGQSSE
YHTACDINYNSQTSIEQCWISGLPDINTEDSAIVSKLNSIVSGWVSDYGFDGLRIDTVKHIRKDFWDGYV
SAAGVFATGEVLSGDVSYVSPYQQHVPSLLNYPLYYPVYDVFTKSRTMSRLSSGFSDIKNGNFKNIDVLV
NFIDNHDQPRLLSKADQSLVKNALAYSFMVQGIPVLYYGTEQSFKGGNDPNNREVLWTTGYSTTSDMYKF
VTTLVKARKGSNSTVNMGIAQTDNVYVFQRGGSLVVVNNYGQGSTNTITVKAGSFSNGDTLTDVFSNKSV
TVQNNQITFQLQNGNPAIFQKK
```

1. A yeast formulation comprising at least one kilogram of a genetically engineered yeast in active dry form.

2. The yeast formulation according to claim 1 comprising at least one recombinant gene for hydrolyzing starch.

3. The yeast formulation according to claim 1 comprising at least one engineered nucleotide change into an endogenous gene.

4. The yeast formulation according to claim 1 comprising a glucoamylase.

5. The yeast formulation according to claim 1 comprising SEQ ID NO: 1 or an enzyme 80%, 85%, 90%, 95%, or 99% identical thereto.

6. The yeast formulation according to claim 1 further comprising at least one additional recombinant gene, wherein the at least one additional recombinant gene encodes an alpha amylase, a glucoamylase, a cutinase, or a trehalase.

7. The yeast formulation according to claim 1 which comprises SEQ ID NO: 2.

8. The yeast formulation according to claim 1 wherein the species is Saccharomyces cerevisiae.

9. The yeast formulation according to claim 1 comprising an additional yeast species.

10. A method of making at least one kilogram of genetically engineered yeast in active dry form comprising;
growing a genetically modified yeast in a fermentation medium comprising at least 10,000 liters;
recovering the yeast wherein no washing is performed; and,
formulating an active dry form yeast, wherein the resulting active dry form yeast maintain equivalent viability compared to a control group in which washing was performed.

11. The method of claim 10 wherein the formulating comprises fluid bed drying.

12. A method of making a desired biochemical comprising including the yeast of claim 1 in a fermentation process with a feedstock, wherein the desired biochemical is selected from the group consisting of ethanol, butanol, etc. arabinitol, n-butanol, isobutanol, ethanol, glycerol, methanol, ethylene glycol, 1,3-propanediol [propylene glycol], butanediol, glycerin, sorbitol, and xylitol); an alkane (e.g., pentane, hexane, heptane, octane, nonane, decane, undecane, and dodecane), a cycloalkane (e.g., cyclopentane, cyclohexane, cycloheptane, and cyclooctane), an alkene (e.g. pentene, hexene, heptene, and octene); an amino acid (e.g., aspartic acid, glutamic acid, glycine, lysine, serine, tryptophan, and threonine); a gas (e.g., methane, hydrogen (H2), carbon
dioxide (CO2), and carbon monoxide (CO)); isoprene, isoprenoid, sesquiterpene; a ketone (e.g., acetone); an aldehyde (e.g., acetaldehyde, butryladehyde); an organic acid (e.g., acetic acid, acetonic acid, adipic acid, ascorbic acid, citric acid, 2,5-diketo-D-gluconic acid, formic acid, fumaric acid, glucaric acid, gluconic acid, glucuronic acid, glutaric acid, 3-hydroxypropionic acid, itaconic acid, lactic acid, malic acid, malonic acid, oxalic acid, oxaloacetic acid, propionic acid, succinic acid, and xylonic acid); 1-3 propane diol, and polyketide.

13. The method of claim 12 wherein the fermentation employs a feedstock selected from the group consisting of glucose, liquefied starch, granular starch, or cellulose.

14. A Saccharomyces cerevisiae yeast comprising SEQ ID NO: 1 or a sequence 90%, 95%, 98%, or 99% identical to it.

15. The Saccharomyces cerevisiae yeast of claim 14 further comprising SEQ ID NO: 2.
